(19) United States
(12) Patent Application Publication
(10) Pub. No.: US 2021/0346259 A1
(43) Pub. Date: Nov. 11, 2021

(54) Oral Care Compositions Comprising Dicarboxylic Acid

(71) Applicant: The Procter & Gamble Company, Cincinnati, OH (US)

(72) Inventors: Michael David Curtis, Mason, OH (US); Andrew Frederic Groth, Mason, OH (US); Paul Albert Sagel, Maineville, OH (US); Samuel James St. John, Cincinnati, OH (US); Marianne Zsiska, Cincinnati, OH (US)

(21) Appl. No.: 17/308,078

(22) Filed: May 5, 2021

Related U.S. Application Data
(60) Provisional application No. 63/020,033, filed on May 5, 2020.

(51) Int. Cl. A61K 8/362; A61Q 11/00; A61K 8/21; A61K 8/41; A61K 8/37; A61K 8/22; A61K 8/73; A61K 8/60; A61K 8/29; A61K 8/44

(52) U.S. Cl. CPC A61K 8/362; A61Q 11/00; A61K 8/21; A61K 8/416; A61K 8/37; A61K 8/22; A61K 2800/74; A61K 8/60; A61K 8/29; A61K 8/44; A61K 2800/28; A61K 2800/48; A61K 8/73

(57) ABSTRACT

Oral care compositions including dicarboxylic acid and a pH of from about 4 to about 6. Oral care kits including a first oral care composition with fluoride and a second oral care composition with dicarboxylic acid. Oral care compositions including fluoride and dicarboxylic acid that provide an erosion benefit, a stain prevention benefit, and/or a stain removal benefit.

### FIELD OF THE INVENTION

The present invention relates to oral care compositions comprising dicarboxylic acid with an improved whitening benefit. The present invention also relates to whitening oral care compositions comprising cationic antimicrobial agents and dicarboxylic acid.

### BACKGROUND OF THE INVENTION

Oral care compositions, such as toothpaste and/or dentifrice compositions, can be applied to the oral cavity to clean and/or maintain the aesthetics and/or health of the teeth, gums, and/or tongue. Additionally, many oral care compositions are can be used to remove and/or prevent stains on oral cavity surfaces. Whitening of oral care hard tissue surfaces can occur through chemical or physical means. Physical agents include the combination of a brush and abrasive. Chemical agents include oxidizing agents (e.g., peroxide), anticalculus agents (e.g., polyphosphates), or other agents capable of dislodging surface stains through chemical action (e.g., bicarbonates).

Each agent has their drawbacks. Oxidizing agents are challenging to keep from reacting with other ingredients of the oral care composition during the composition's lifecycle. Additionally, they are not reactive with some surface stains; thereby, not fulfilling their primary purpose. Abrasive agents can cause damage to oral hard tissue surface. Furthermore, they cannot access all areas of the tooth surface where there are stains (e.g., interproximal spaces). Polyphosphate-based anticalculus agents are highly susceptible to hydrolysis breaking down in compositions to ineffective orthophosphate. In the presence of soluble fluoride, the breakdown can be accelerated resulting in insoluble fluoride. Some other chemical agents have characteristic tastes that make them unpleasant to consumers. Bicarbonate-based toothpastes tend to taste like baking soda whose unique experience is not enjoyed by a wide slice of consumers. In total, existing whitening agents can be challenging to formulate with for a variety of reasons specific to each agent.

Thus, there is a need for a whitening agent that can effectively remove and prevent the accumulation of stain, while improving existing formulation challenges.

### SUMMARY OF THE INVENTION

Disclosed herein is an oral care composition comprising (a) dicarboxylic acid; and (b) a pH of from about 4 to about 6, wherein the oral care composition has a pellicle cleaning ratio of at least about 120.

Also disclosed herein is a whitening dentifrice composition comprising (a) dicarboxylic acid; and (b) a pH of from about 4 to about 6, wherein the whitening dentifrice composition has a pellicle cleaning ratio of at least about 120.

Also disclosed herein is an oral care kit comprising (a) a first oral care composition comprising fluoride; and (b) a second oral care composition comprising dicarboxylic acid.

Also disclosed herein is an oral care kit comprising (a) a first oral care composition comprising tin; and (b) a second oral care composition comprising dicarboxylic acid.

Also disclosed herein is a method for whitening teeth comprising instructing a user to apply any one of the compositions disclosed herein to the oral cavity and/or teeth.

Also disclosed herein is a regimen for whitening teeth comprising (a) applying a dentifrice composition comprising fluoride and/or tin for a first duration of from about 30 seconds to about 2 minutes, (b) expectorating the dentifrice composition; and (c) applying a whitening composition comprising dicarboxylic acid for a second duration of from about 30 seconds to about 2 minutes.

Also disclosed herein is a whitening dentifrice composition comprising (a) dicarboxylic acid; and (b) a pH of from about 4 to about 6, wherein the whitening dentifrice composition has an increase in its pellicle cleaning ratio relative to its pH-matched, dicarboxylic-acid placebo by a factor of at least about 1.2.

### DETAILED DESCRIPTION OF THE INVENTION

The present invention is directed to oral care whitening compositions that have oxalate and provide an unexpectedly high stain removal benefit relative to other conventional chemical stain removal agents in a particular pH range. Dental stain, or tooth stain, is caused by the cation-crosslinked proteins and extracellular polysaccharides that then act as reservoirs for colored porphyrins and organic and/or inorganic chromophores. Cross-linking can occur electrostatically via charge-charge, dipole-dipole, and/or dipole-charge interactions. Interrupting these electrostatic forces can facilitate stain removal. The resulting invention provides efficacious oral hard tissue whitening benefits with fewer drawbacks that are observed with other whitening agents.

Chemical whitening agents loosen the bonds of this colored matrix to affect its removal from the oral hard tissue surfaces. While not wishing to be bound by theory, chemical agents that are effective solubilizing ligands of cationic crosslinking agents in the colored matrix on the oral hard tissue surfaces can be used to remove stain from the surface. Furthermore, the pH and ionic strength of the oral care composition can be used to reduce the strength electrostatic bonds by protonating anionically charged moieties or by reducing the potential of the electrostatic double layer further facilitating the solubilization of cationic moieties by solubilizing ligands (i.e., whitening agents).

The chelate effect postulates that complexes of polydentate ligands with a metal are more stable than the dentate-normalized equivalent of the monodentate-ligand-stabilized metal complex (e.g., 1 mole of a bidentate ligand in comparison to 2 moles of a similarly structured monodentate ligand) because of a reduction in molar entropy of the bidentate chelate with respect to the monodentate complex. The unique properties of oxalate anion allows it, therefore, to be a highly effective stabilizing ligand in a particular pH range. In this way, the oxalate anion in a particular pH range is capable of solubilizing and excising metal cations from stained oral enamel and dentin surfaces allowing for facile removal of stained chromogens. While not wishing to be bound by theory, it is believed that the disclosed oral care compositions of the present invention provide an unexpectedly high whitening benefit in comparison to a conventional whitening agent, pyrophosphate.

Oxalate anion can extract calcium from the enamel mineral in order to form this insoluble phase. Until the insoluble phase is formed, the oxalate enhances the surface solubility of the enamel surface by reducing the local degree of saturation of enamel with respect to calcium. At low pH and low calcium content (e.g., during exposure to the oral care composition), the application of the oxalate anion may result in measurable softening of the enamel surface. It was unexpectedly discovered during the application of the low pH, oxalate-containing oral care composition to whiten later resulted in measurable surface demineralization not previously disclosed in the art. Consequently, the lower pH of oxalate-containing oral care compositions can be at least about 4.5 to avoid measurable softening of the enamel surface occurs that may result in damage if used routinely.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied.

The term “oral care composition”, as used herein, includes a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces.

The term “dentifrice composition”, as used herein, includes tooth or subgingival-paste, gel, or liquid formulations unless otherwise specified. The dentifrice composition may be a single-phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

“Active and other ingredients” useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated function(s) or activities listed.

The term “orally acceptable carrier” comprises one or more compatible solid or liquid excipients or diluents which are suitable for topical oral administration. By “compatible,” as used herein, is meant that the components of the composition are capable of being commingled without interaction in a manner which would substantially reduce the composition's stability and/or efficacy. The carriers or excipients of the present invention can include the usual and conventional components of mouthwashes or mouth rinses, as more fully described hereinafter: Mouthwash or mouth rinse carrier materials typically include, but are not limited to one or more of water, alcohol, humectants, surfactants, and acceptance improving agents, such as flavoring, sweetening, coloring and/or cooling agents.

The term “substantially free” as used herein refers to the presence of no more than 0.05%, preferably no more than 0.01%, and more preferably no more than 0.001%, of an indicated material in a composition, by total weight of such composition.

The term “essentially free” as used herein means that the indicated material is not deliberately added to the composition, or preferably not present at analytically detectable levels. It is meant to include compositions whereby the indicated material is present only as an impurity of one of the other materials deliberately added.

The term “oral hygiene regimen” or “regimen” can be for the use of two or more separate and distinct treatment steps for oral health. e.g. toothpaste, mouth rinse, floss, toothpicks, spray, water irrigator, massager.

The term “total water content” as used herein means both free water and water that is bound by other ingredients in the oral care composition.

For the purpose of the present invention, the relevant molecular weight (MW) to be used is that of the material added when preparing the composition e.g., if the chelant is a citrate species, which can be supplied as citric acid, sodium citrate or indeed other salt forms, the MW used is that of the particular salt or acid added to the composition but ignoring any water of crystallization that may be present.

While compositions and methods are described herein in terms of “comprising” various components or steps, the compositions and methods can also “consist essentially of” or “consist of” the various components or steps, unless stated otherwise.

As used herein, the word “or” when used as a connector of two or more elements is meant to include the elements individually and in combination; for example, X or Y, means X or Y or both.

As used herein, the articles “a” and “an” are understood to mean one or more of the material that is claimed or described, for example, “an oral care composition” or “a bleaching agent.”

All measurements referred to herein are made at about 23° C. (i.e. room temperature) unless otherwise specified.

Generally, groups of elements are indicated using the numbering scheme indicated in the version of the periodic table of elements published in Chemical and Engineering News, 63(5), 27, 1985. In some instances, a group of elements can be indicated using a common name assigned to the group; for example, alkali metals for Group 1 elements, alkaline earth metals for Group 2 elements, and so forth.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein.

The term “about” means that amounts, sizes, formulations, parameters, and other quantities and characteristics are not and need not be exact, but can be approximate and/or larger or smaller, as desired, reflecting tolerances, conversion factors, rounding off, measurement errors, and the like, and other factors known to those of skill in the art. In general, an amount, size, formulation, parameter or other quantity or characteristic is “about” or “approximate” whether or not expressly stated to be such. The term “about” also encompasses amounts that differ due to different equilibrium conditions for a composition resulting from a particular initial mixture. Whether or not modified by the term “about,” the claims include equivalents to the quantities. The term “about” can mean within 10% of the reported numerical value, preferably within 5% of the reported numerical value.

The dentifrice composition can be in any suitable form, such as a solid, liquid, powder, paste, or combinations thereof. The oral care composition can be dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The components of the dentifrice composition can be incorporated into a film, a strip, a foam, or a fiber-based dentifrice composition.

The oral care compositions, as described herein, comprise dicarboxylic acid, tin, and/or fluoride. Additionally, the oral care compositions can comprise other optional ingredients, as described below. The section headers below are provided for convenience only. In some cases, a compound can fall within one or more sections. For example, stannous fluoride can be a tin compound and/or a fluoride compound. Additionally, oxalic acid, or salts thereof, can be a dicarboxylic acid, a polydentate ligand, and/or a whitening agent.

Dicarboxylic Acid

The oral care composition comprises dicarboxylic acid. The dicarboxylic acid comprises a compound with two carboxylic acid functional groups. The dicarboxylic acid can comprise a compound or salt thereof defined by Formula I.

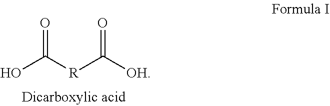

R can be null, alkyl, alkenyl, allyl, phenyl, benzyl, aliphatic, aromatic, polyethylene glycol, polymer, O, N, P, or combinations thereof.

The dicarboxylic acid can comprise oxalic acid, malonic acid, succinic acid, glutaric acid, adipic acid, pimelic acid, suberic acid, azerlaic acid, sebacic acid, undecanedioic acid, dodecanedioic acid, bras sylic acid, thap sic acid, japanic acid, phellogenic acid, equisetolic acid, malic acid, maleic acid, tartaric acid, phthalic acid, methylmalonic acid, dimethylmalonic acid, tartronic acid, mesoxalic acid, dihydroxymalonic acid, fumaric acid, terephthalic acid, glutaric acid, salts thereof, or combinations thereof. The dicarboxylic acid can comprise suitable salts of dicarboxylic acid, such as, for example, monoalkali metal oxalate, dialkali metal oxalate, monopotassium monohydrogen oxalate, dipotassium oxalate, monosodium monohydrogen oxalate, disodium oxalate, titanium oxalate, and/or other metal salts of oxalate. The dicarboxylic acid can also include hydrates of the dicarboxylic acid and/or a hydrate of a salt of the dicarboxylic acid.

The oral care composition can comprise from about 0.01% to about 10%, from about 0.1% to about 15%, from about 1% to about 5%, or from about 0.0001 to about 25%, of dicarboxylic acid.

Fluoride

The oral care composition can comprise fluoride, which can be provided by a fluoride ion source. The fluoride ion source can comprise one or more fluoride containing compounds, such as stannous fluoride, sodium fluoride, potassium fluoride, amine fluoride, sodium monofluorophosphate, zinc fluoride, and/or mixtures thereof.

The fluoride ion source and the tin ion source can be the same compound, such as for example, stannous fluoride, which can generate tin ions and fluoride ions. Additionally, the fluoride ion source and the tin ion source can be separate compounds, such as when the tin ion source is stannous chloride and the fluoride ion source is sodium monofluorophosphate or sodium fluoride.

The fluoride ion source and the zinc ion source can be the same compound, such as for example, zinc fluoride, which can generate zinc ions and fluoride ions. Additionally, the fluoride ion source and the zinc ion source can be separate compounds, such as when the zinc ion source is zinc phosphate and the fluoride ion source is stannous fluoride.

The fluoride ion source can be essentially free of, or free of stannous fluoride. Thus, the oral care composition can comprise sodium fluoride, potassium fluoride, amine fluoride, sodium monofluorophosphate, zinc fluoride, and/or mixtures thereof.

The oral care composition can comprise a fluoride ion source capable of providing from about 50 ppm to about 5000 ppm, and preferably from about 500 ppm to about 3000 ppm of free fluoride ions. To deliver the desired amount of fluoride ions, the fluoride ion source may be present in the oral care composition at an amount of from about 0.0025% to about 5%, from about 0.01% to about 10%, from about 0.2% to about 1%, from about 0.5% to about 1.5%, or from about 0.3% to about 0.6%, by weight of the oral care composition. Alternatively, the oral care composition can comprise less than 0.1%, less than 0.01%, be essentially free of, be substantially free of, or free of a fluoride ion source.

Metal

The oral care composition, as described herein, can comprise metal, which can be provided by a metal ion source comprising one or more metal ions. The metal ion source can comprise or be in addition to the tin ion source and/or the zinc ion source, as described herein. Suitable metal ion sources include compounds with metal ions, such as, but not limited to Sn, Zn, Cu, Mn, Mg, Sr, Ti, Fe, Mo, B, Ba, Ce, Al, In and/or mixtures thereof. The metal ion source can be any compound with a suitable metal and any accompanying ligands and/or anions.

Suitable ligands and/or anions that can be paired with metal ion sources include, but are not limited to acetate, ammonium sulfate, benzoate, bromide, borate, carbonate, chloride, citrate, gluconate, glycerophosphate, hydroxide, iodide, oxalate, oxide, propionate, D-lactate, DL-lactate, orthophosphate, pyrophosphate, sulfate, nitrate, tartrate, and/or mixtures thereof.

The oral care composition can comprise from about 0.01% to about 10%, from about 1% to about 5%, or from about 0.5% to about 15% of metal and/or a metal ion source.

Tin

The oral care composition of the present invention can comprise tin, which can be provided by a tin ion source. The tin ion source can be any suitable compound that can provide tin ions in an oral care composition and/or deliver tin ions to the oral cavity when the oral care composition is applied to the oral cavity. The tin ion source can comprise one or more tin containing compounds, such as stannous fluoride, stannous chloride, stannous bromide, stannous iodide, stannous oxide, stannous oxalate, stannous sulfate, stannous sulfide, stannic fluoride, stannic chloride, stannic bromide, stannic iodide, stannic sulfide, and/or mixtures thereof. Tin ion source can comprise stannous fluoride, stannous chloride, and/or mixture thereof. The tin ion source can also be a fluoride-free tin ion source, such as stannous chloride.

The oral care composition can comprise from about 0.0025% to about 5%, from about 0.01% to about 10%, from about 0.2% to about 1%, from about 0.4% to about 1%, or from about 0.3% to about 0.6%, by weight of the oral care composition, of tin and/or a tin ion source. Alternatively, the oral care composition can be essentially free of, substantially free of, or free of tin.

Zinc

The oral care composition can comprise zinc, which can be provided by a zinc ion source. The zinc ion source can comprise one or more zinc containing compounds, such as zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, and/or zinc carbonate. The zinc ion source can be a fluoride-free zinc ion source, such as zinc phosphate, zinc oxide, and/or zinc citrate.

The zinc and/or zinc ion source may be present in the total oral care composition at an amount of from about 0.01% to about 10%, from about 0.2% to about 1%, from about 0.4% to about 1%, or from about 0.3% to about 0.6%, by weight of the dentifrice composition. Alternatively, the oral care composition can be essentially free of, substantially free of, or free of zinc.

pH

The pH of the oral care compositions as described herein can be from about 4 to about 7, from about 4 to about 6, from about 4.5 to about 6.5, or from about 4.5 to about 5.5. The pH of a mouthrinse solution can be determined as the pH of the neat solution. The pH of a dentifrice composition can be determined as a slurry pH, which is the pH of a mixture of the dentifrice composition and water, such as a 1:4, 1:3, or 1:2 mixture of the dentifrice composition and water.

The pH of the oral care compositions as described herein have a preferred pH of below about 7 or below about 6 due to the pKa of the dicarboxylic acid. While not wishing to be bound by theory, it is believed that the dicarboxylic acid displays unique behavior when the pH is below about 7 or below about 6, but surfaces in the oral cavity can also be sensitive to a low pH. Additionally, at pH values above about pH 7, the metal ion source can react with water and/or hydroxide ions to form insoluble metal oxides and/or metal hydroxides. The formation of these insoluble compounds can limit the ability of dicarboxylates to stabilize metal ions in oral care compositions and/or can limit the interaction of dicarboxylates with target metal ions in the oral cavity.

Additionally, at pH values less than 4, the potential to damage teeth by acid dissolution is greatly increased. Consequently, the oral care compositions comprising dicarboxylic acid, as described herein, preferably have a pH from about 4 to about 7, from about 4 to about 6, from about 4.5 to about 6.5, or from about 4.5 to about 5.5 to minimize metal hydroxide/metal oxide formation and any damage to oral hard tissues (enamel, dentin, and cementum).

The oral care composition can comprise one or more buffering agents. Buffering agents, as used herein, refer to agents that can be used to adjust the slurry pH of the oral care compositions. The buffering agents include alkali metal hydroxides, carbonates, sesquicarbonates, borates, silicates, phosphates, imidazole, and mixtures thereof. Specific buffering agents include monosodium phosphate, trisodium phosphate, sodium hydroxide, potassium hydroxide, alkali metal carbonate salts, sodium carbonate, imidazole, pyrophosphate salts, citric acid, and sodium citrate. The oral care composition can comprise one or more buffering agents each at a level of from about 0.1% to about 30%, from about 1% to about 10%, or from about 1.5% to about 3%, by weight of the present composition.

Polyphosphate

The oral care composition can comprise polyphosphate, which can be provided by a polyphosphate source. A polyphosphate source can comprise one or more polyphosphate molecules. Polyphosphates are a class of materials obtained by the dehydration and condensation of orthophosphate to yield linear and cyclic polyphosphates of varying chain lengths. Thus, polyphosphate molecules are generally identified with an average number (n) of polyphosphate molecules, as described below. A polyphosphate is generally understood to consist of two or more phosphate molecules arranged primarily in a linear configuration, although some cyclic derivatives may be present.

Preferred polyphosphates are those having an average of two or more phosphate groups so that surface adsorption at effective concentrations produces sufficient non-bound phosphate functions, which enhance the anionic surface charge as well as hydrophilic character of the surfaces. Preferred in this invention are the linear polyphosphates having the formula: XO(XPO₃)ₙX, wherein X is sodium, potassium, ammonium, or any other alkali metal cations and n averages from about 2 to about 21. Alkali earth metal cations, such as calcium, are not preferred because they tend to form insoluble fluoride salts from aqueous solutions comprising a fluoride ions and alkali earth metal cations. Thus, the oral care compositions disclosed herein can be free of or substantially free of calcium pyrophosphate.

Some examples of suitable polyphosphate molecules include, for example, pyrophosphate (n=2), tripolyphosphate (n=3), tetrapolyphosphate (n=4), sodaphos polyphosphate (n=6), hexaphos polyphosphate (n=13), benephos polyphosphate (n=14), hexarrietaphosphate (n=21), which is also known as Glass H. Polyphosphates can include those polyphosphate compounds manufactured by FMC Corporation, ICL Performance Products, and/or Astaris.

The oral care composition can comprise from about 0.01% to about 15%, from about 0.1% to about 10%, from about 0.5% to about 5%, from about 1 to about 20%, or about 10% or less, by weight of the oral care composition, of the polyphosphate source. Alternatively, the oral care composition can be essentially free of, substantially free of, or free of polyphosphate.

Surfactants

The oral care composition can comprise one or more surfactants. The surfactants can be used to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants, such as sodium lauryl sulfate, sodium lauryl isethionate, sodium lauroyl methyl isethionate, sodium cocoyl glutamate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine, sodium cocoyl glutamate, and the like. Sodium lauryl sulfate is a preferred surfactant. The oral care composition can comprise one or more surfactants each at a level from about 0.01% to about 15%, from about 0.3% to about 10%, or from about 0.3% to about 2.5%, by weight of the oral care composition.

Monodentate Ligand

The oral care composition can comprise monodentate ligand having a molecular weight (MW) of less than 1000 g/mol. A monodentate ligand has a single functional group that can interact with the central atom, such as a tin ion. The monodentate ligand must be suitable for the use in oral care composition, which can be include being listed in Generally Regarded as Safe (GRAS) list with the United States Food and Drug Administration or other suitable list in a jurisdiction of interest.

The monodentate ligand, as described herein, can include a single functional group that can chelate to, associate with, and/or bond to tin. Suitable functional groups that can chelate to, associate with, and/or bond to tin include carbonyl, amine, among other functional groups known to a person of ordinary skill in the art. Suitable carbonyl functional groups can include carboxylic acid, ester, amide, or ketones.

The monodentate ligand can comprise a single carboxylic acid functional group. Suitable monodentate ligands comprising carboxylic acid can include compounds with the formula R—COOH, wherein R is any organic structure. Suitable monodentate ligands comprising carboxylic acid can also include aliphatic carboxylic acid, aromatic carboxylic acid, sugar acid, salts thereof, and/or combinations thereof.

The aliphatic carboxylic acid can comprise a carboxylic acid functional group attached to a linear hydrocarbon chain, a branched hydrocarbon chain, and/or cyclic hydrocarbon molecule. The aliphatic carboxylic acid can be fully saturated or unsaturated and have one or more alkene and/or alkyne functional groups. Other functional groups can be present and bonded to the hydrocarbon chain, including halogenated variants of the hydrocarbon chain. The aliphatic carboxylic acid can also include hydroxyl acids, which are organic compounds with an alcohol functional group in the alpha, beta, or gamma position relative to the carboxylic acid functional group. A suitable alpha hydroxy acid includes lactic acid and/or a salt thereof.

The aromatic carboxylic acid can comprise a carboxylic acid functional group attached to at least one aromatic functional group. Suitable aromatic carboxylic acid groups can include benzoic acid, salicylic acid, and/or combinations thereof.

The carboxylic acid can include formic acid, acetic acid, propionic acid, butyric acid, valeric acid, caproic acid, enanthic acid, caprylic acid, ascorbic acid, benzoic acid, caprylic acid, cholic acid, glycine, alanine, valine, isoleucine, leucine, phenylalanine, linoleic acid, niacin, oleic acid, propanoic acid, sorbic acid, stearic acid, gluconate, lactate, carbonate, chloroacetic acid, dichloroacetic acid, trichloroacetic acid, salts thereof, and/or combinations thereof.

The oral care composition can include from about 0.01% to about 10%, from about 0.1% to about 15%, from about 1% to about 5%, or from about 0.0001 to about 25%, by weight of the composition, of the monodentate ligand.

Polydentate Ligand

The oral care composition can comprise polydentate ligand having a molecular weight (MW) of less than 1000 g/mol or less than 2500 g/mol. A polydentate ligand has at least two functional groups that can interact with the central atom, such as a tin ion. Additionally, the polydentate ligand must be suitable for the use in oral care composition, which can be include being listed in Generally Regarded as Safe (GRAS) list with the United States Food and Drug Administration or another suitable list in a jurisdiction of interest.

The polydentate ligand, as described herein, can include at least two functional groups that can chelate to, associate with, and/or bond to tin. The polydentate ligand can comprise a bidentate ligand (i.e. with two functional groups), tridentate (i.e. with three functional groups), tetradentate (i.e. with four functional groups), etc.

Suitable functional groups that can chelate to, associate with, and/or bond to tin include carbonyl, phosphate, nitrate, amine, among other functional groups known to a person of ordinary skill in the art. Suitable carbonyl functional groups can include carboxylic acid, ester, amide, or ketones.

The polydentate ligand can comprise two or more carboxylic acid functional groups. Suitable polydentate ligands comprising carboxylic acid can include compounds with the formula HOOC—R—COOH, wherein R is any organic structure. Suitable polydentate ligands comprising two or more carboxylic acid can also include dicarboxylic acid, tricarboxylic acid, tetracarboxylic acid, etc.

Other suitable polydentate ligands include compounds comprising at least two phosphate functional groups. Thus, the polydentate ligand can comprise polyphosphate, as described herein.

Other suitable polydentate ligands include hops beta acids, such as lupulone, colupulone, adlupulone, and/or combinations thereof. The hops beta acid can be synthetically derived and/or extracted from a natural source.

The polydentate ligand can also include phosphate as the functional group to interact with the tin. Suitable phosphate compounds include phosphate salts, organophosphates, or combinations thereof. Suitable phosphate salts include salts of orthophosphate, hydrogen phosphate, dihydrogen phosphate, alkylated phosphates, and combinations thereof. The polydentate ligand can comprise oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, maleic acid, malic acid, tartaric acid, phthalic acid, citric acid, phytic acid, pyrophosphate, tripolyphosphate, tetrapolyphosphate, hexametaphosphate, salts thereof, and/or combinations thereof.

The oral care composition can include from about 0.01% to about 10%, from about 0.1% to about 15%, from about 1% to about 5%, or from about 0.0001 to about 25%, by weight of the composition, of the polydentate ligand.

Ratio of Tin to Monodentate Ligand to Polydentate Ligand

The oral care composition, as described herein, can comprise a ratio of tin to monodentate ligand to polydentate ligand that provides an unexpectedly high amount of soluble tin and/or a superior fluoride uptake. Suitable ratios of tin to monodentate ligand to polydentate ligand can be from about 1:0.5:0.5 to about 1:5:5, from about 1:0.5:0.75 to about 1:5:5, from about 1:1:1 to about 1:5:5, from about 1:1:0.5 to about 1:2.5:2.5, from about 1:1:1 to about 1:2:2, from about 1:0.5:0.5 to about 1:3:1, or from about 1:0.5:0.5 to about 1:1:3.

Desired herein are oral care compositions with a soluble Sn of at least about 1000 ppm, 2000 ppm, 4000 ppm, at least about 4500 ppm, at least about 5000 ppm, at least about 6000 ppm, and/or at least about 8000 ppm. Also desired herein are oral care compositions with a fluoride uptake of at least about 6.5 μg/cm², at least about 7.0 μg/cm², at least about 8.0 μg/cm², or at least about 9.0 μg/cm² after a time period of at least about 9 days, 30 days, 65 days, 75 days, 100 days, 200 days, 365 days and/or 400 days.

In total, while not wishing to be bound by theory it is believed that the soluble Sn amount is correlated to bioavailable Sn as it is freely available to provide an oral health benefit. Fully bound Sn (i.e. Sn that is overchelated) or precipitated Sn (i.e. insoluble tin salts, such as Sn(OH)₂ and/or Sn-based stains can form when Sn is underchelated) would not be included in the measurement for soluble Sn. Additionally, while not wishing to be bound by theory, it is believed that a carefully balanced ratio of Sn to monodentate and polydentate ligands can provide a high amount of bioavailable fluoride and Sn ions without some of the negatives to the use of cationic antimicrobial agents, such as surface staining. Thus, additional screening experiments were done to quantify and qualify the ranges and identities of monodentate and polydentate ligands.

Thickening Agent

The oral care composition can comprise one or more thickening agents. Thickening agents can be useful in the oral care compositions to provide a gelatinous structure that stabilizes the toothpaste against phase separation. Suitable thickening agents include polysaccharides, polymers, and/or silica thickeners. Some non-limiting examples of polysaccharides include starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof.

The thickening agent can comprise polysaccharides. Polysaccharides that are suitable for use herein include carageenans, gellan gum, locust bean gum, xanthan gum, carbomers, poloxamers, modified cellulose, and mixtures thereof. Carageenan is a polysaccharide derived from seaweed. There are several types of carageenan that may be distinguished by their seaweed source and/or by their degree of and position of sulfation. The thickening agent can comprise kappa carageenans, modified kappa carageenans, iota carageenans, modified iota carageenans, lambda carrageenan, and mixtures thereof. Carageenans suitable for use herein include those commercially available from the FMC Company under the series designation “Viscarin,” including but not limited to Viscarin TP 329, Viscarin TP 388, and Viscarin TP 389.

The thickening agent can comprise one or more polymers. The polymer can be a polyethylene glycol (PEG), a polyvinylpyrrolidone (PVP), polyacrylic acid, a polymer derived from at least one acrylic acid monomer, a copolymer of maleic anhydride and methyl vinyl ether, a crosslinked polyacrylic acid polymer, of various weight percentages of the oral care composition as well as various ranges of average molecular ranges. The polymer can comprise polyacrylate crosspolymer, such as polyacrylate crosspolymer-6. Suitable sources of polyacrylate crosspolymer-6 can include Sepimax Zen™ commercially available from Seppic.

The thickening agent can comprise inorganic thickening agents. Some non-limiting examples of suitable inorganic thickening agents include colloidal magnesium aluminum silicate, silica thickeners. Useful silica thickeners include, for example, include, as a non-limiting example, an amorphous precipitated silica such as ZEODENT® 165 silica. Other non-limiting silica thickeners include ZEODENT® 153, 163, and 167, and ZEOFREE® 177 and 265 silica products, all available from Evonik Corporation, and AEROSIL® fumed silicas. The oral care composition can comprise from 0.01% to about 15%, from 0.1% to about 10%, from about 0.2% to about 5%, or from about 0.5% to about 2% of one or more thickening agents.

Abrasive

The oral care composition of the present invention can comprise an abrasive. Abrasives can be added to oral care formulations to help remove surface stains from teeth. Preferably, the abrasive is a calcium abrasive or a silica abrasive.

The calcium abrasive can be any suitable abrasive compound that can provide calcium ions in an oral care composition and/or deliver calcium ions to the oral cavity when the oral care composition is applied to the oral cavity. The oral care composition can comprise from about 5% to about 70%, from about 10% to about 60%, from about 20% to about 50%, from about 25% to about 40%, or from about 1% to about 50% of a calcium abrasive. The calcium abrasive can comprise one or more calcium abrasive compounds, such as calcium carbonate, precipitated calcium carbonate (PCC), ground calcium carbonate (GCC), chalk, dicalcium phosphate, calcium pyrophosphate, and/or mixtures thereof.

The oral care composition can also comprise a silica abrasive, such as silica gel (by itself, and of any structure), precipitated silica, amorphous precipitated silica (by itself, and of any structure as well), hydrated silica, and/or combinations thereof. The oral care composition can comprise from about 5% to about 70%, from about 10% to about 60%, from about 10% to about 50%, from about 20% to about 50%, from about 25% to about 40%, or from about 1% to about 50% of a silica abrasive.

The oral care composition can also comprise another abrasive, such as bentonite, perlite, titanium dioxide, alumina, hydrated alumina, calcined alumina, aluminum silicate, insoluble sodium metaphosphate, insoluble potassium metaphosphate, insoluble magnesium carbonate, zirconium silicate, particulate thermosetting resins and other suitable abrasive materials. The oral care composition can comprise from about 5% to about 70%, from about 10% to about 60%, from about 10% to about 50%, from about 20% to about 50%, from about 25% to about 40%, or from about 1% to about 50% of another abrasive.

Amino Acid

The oral care composition can comprise amino acid. The amino acid can comprise one or more amino acids, peptide, and/or polypeptide, as described herein.

Amino acids, as in Formula II, are organic compounds that contain an amine functional group, a carboxyl functional group, and a side chain (R in Formula II) specific to each amino acid. Suitable amino acids include, for example, amino acids with a positive or negative side chain, amino acids with an acidic or basic side chain, amino acids with polar uncharged side chains, amino acids with hydrophobic side chains, and/or combinations thereof. Suitable amino acids also include, for example, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutanoic acid, diaminoproprionic acid, salts thereof, and/or combinations thereof.

Suitable amino acids include the compounds described by Formula II, either naturally occurring or synthetically derived. The amino acid can be zwitterionic, neutral, positively charged, or negatively charged based on the R group and the environment. The charge of the amino acid, and whether particular functional groups, can interact with tin at particular pH conditions, would be well known to one of ordinary skill in the art.

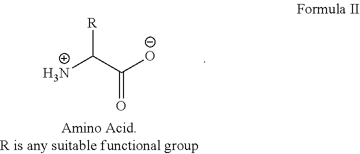

Suitable amino acids include one or more basic amino acids, one or more acidic amino acids, one or more neutral amino acids, or combinations thereof.

The oral care composition can comprise from about 0.01% to about 20%, from about 0.1% to about 10%, from about 0.5% to about 6%, or from about 1% to about 10% of amino acid, by weight of the oral care composition.

The term “neutral amino acids” as used herein include not only naturally occurring neutral amino acids, such as alanine, asparagine, cysteine, glutamine, glycine, isoleucine, leucine, methionine, phenylalanine, proline, serine, threonine, tryptophan, tyrosine, valine, but also biologically acceptable amino acids which have an isoelectric point in range of pH 5.0 to 7.0. The biologically preferred acceptable neutral amino acid has a single amino group and carboxyl group in the molecule or a functional derivative hereof, such as functional derivatives having an altered side chain albeit similar or substantially similar physio chemical properties. In a further embodiment the amino acid would be at minimum partially water soluble and provide a pH of less than 7 in an aqueous solution of 1 g/1000 ml at 25° C.

Accordingly, neutral amino acids suitable for use in the invention include, but are not limited to, alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, salts thereof, or mixtures thereof. Preferably, neutral amino acids used in the composition of the present invention may include asparagine, glutamine, glycine, salts thereof, or mixtures thereof. The neutral amino acids may have an isoelectric point of 5.0, or 5.1, or 5.2, or 5.3, or 5.4, or 5.5, or 5.6, or 5.7, or 5.8, or 5.9, or 6.0, or 6.1, or 6.2, or 6.3, or 6.4, or 6.5, or 6.6, or 6.7, or 6.8, or 6.9, or 7.0, in an aqueous solution at 25° C. Preferably, the neutral amino acid is selected from proline, glutamine, or glycine, more preferably in its free form (i.e. uncomplexed). If the neutral amino acid is in its salt form, suitable salts include salts known in the art to be pharmaceutically acceptable salts considered to be physiologically acceptable in the amounts and concentrations provided.

Whitening Agent

The oral care composition may comprise from about 0.1% to about 10%, from about 0.2% to about 5%, from about 1% to about 5%, or from about 1% to about 15%, by weight of the oral care composition, of a whitening agent. The whitening agent can be a compound suitable for whitening at least one tooth in the oral cavity. The whitening agent may include peroxides, metal chlorites, perborates, percarbonates, peroxyacids, persulfates, dicarboxylic acids, and combinations thereof. Suitable peroxides include solid peroxides, hydrogen peroxide, urea peroxide, calcium peroxide, benzoyl peroxide, sodium peroxide, barium peroxide, inorganic peroxides, hydroperoxides, organic peroxides, and mixtures thereof. Suitable metal chlorites include calcium chlorite, barium chlorite, magnesium chlorite, lithium chlorite, sodium chlorite, and potassium chlorite. Other suitable whitening agents include sodium persulfate, potassium persulfate, peroxydone, 6-phthalimido peroxy hexanoic acid, pthalamidoperoxycaproic acid, or mixtures thereof.

Humectant

The oral care composition can comprise one or more humectants, have low levels of a humectant, or be free of a humectant. Humectants serve to add body or “mouth texture” to an oral care composition or dentifrice as well as preventing the dentifrice from drying out. Suitable humectants include polyethylene glycol (at a variety of different molecular weights), propylene glycol, glycerin (glycerol), erythritol, xylitol, sorbitol, mannitol, butylene glycol, lactitol, hydrogenated starch hydrolysates, and/or mixtures thereof. The oral care composition can comprise one or more humectants each at a level of from 0 to about 70%, from about 5% to about 50%, from about 10% to about 60%, or from about 20% to about 80%, by weight of the oral care composition.

Water

The oral care composition of the present invention can be a dentifrice composition that is anhydrous, a low water formulation, or a high water formulation. In total, the oral care composition can comprise from 0% to about 99%, about 20% or greater, about 30% or greater, about 50% or greater, up to about 45%, or up to about 75%, by weight of the composition, of water. Preferably, the water is USP water.

In a high water dentifrice formulation, the dentifrice composition comprises from about 45% to about 75%, by weight of the composition, of water. The high water dentifrice composition can comprise from about 45% to about 65%, from about 45% to about 55%, or from about 46% to about 54%, by weight of the composition, of water. The water may be added to the high water dentifrice formulation and/or may come into the composition from the inclusion of other ingredients.

In a low water dentifrice formulation, the dentifrice composition comprises from about 10% to about 45%, by weight of the composition, of water. The low water dentifrice composition can comprise from about 10% to about 35%, from about 15% to about 25%, or from about 20% to about 25%, by weight of the composition, of water. The water may be added to the low water dentifrice formulation and/or may come into the composition from the inclusion of other ingredients.

In an anhydrous dentifrice formulation, the dentifrice composition comprises less than about 10%, by weight of the composition, of water. The anhydrous dentifrice composition comprises less than about 5%, less than about 1%, or 0%, by weight of the composition, of water. The water may be added to the anhydrous formulation and/or may come into the dentifrice composition from the inclusion of other ingredients.

The dentifrice composition can also comprise other orally acceptable carrier materials, such as alcohol, humectants, polymers, surfactants, and acceptance improving agents, such as flavoring, sweetening, coloring and/or cooling agents.

The oral care composition can also be a mouth rinse formulation. A mouth rinse formulation can comprise from about 75% to about 99%, from about 75% to about 95%, or from about 80% to about 95% of water.

Other Ingredients

The oral care composition can comprise a variety of other ingredients, such as flavoring agents, sweeteners, colorants, preservatives, buffering agents, or other ingredients suitable for use in oral care compositions, as described below.

Flavoring agents also can be added to the oral care composition. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, and mixtures thereof. Coolants may also be part of the flavor system. Preferred coolants in the present compositions are the paramenthan carboxyamide agents such as N-ethyl-p-menthan-3-carboxamide (known commercially as “WS-3”) or N-(Ethoxycarbonylmethyl)-3-p-menthanecarboxamide (known commercially as “WS-5”), and mixtures thereof. A flavor system is generally used in the compositions at levels of from about 0.001% to about 5%, by weight of the oral care composition. These flavoring agents generally comprise mixtures of aldehydes, ketones, esters, phenols, acids, and aliphatic, aromatic and other alcohols. Sweeteners can be added to the oral care composition to impart a pleasing taste to the product.

Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, sucralose, stevia, and glucose.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO₂, and colors such as FD&C and D&C dyes.

Preservatives also can be added to the oral care compositions to prevent bacterial growth. Suitable preservatives approved for use in oral compositions such as methylparaben, propylparaben, benzoic acid, and sodium benzoate can be added in safe and effective amounts.

Titanium dioxide may also be added to the present composition. Titanium dioxide is a white powder which adds opacity to the compositions. Titanium dioxide generally comprises from about 0.25% to about 5%, by weight of the oral care composition.

Other ingredients can be used in the oral care composition, such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

Oral Care Composition Forms

Suitable compositions for the delivery of the dicarboxylic acid include emulsion compositions, such as the emulsions compositions of U.S. Patent Application Publication No. 2018/0133121, which is herein incorporated by reference in its entirety, unit-dose compositions, such as the unit-dose compositions of U.S. Patent Application Publication No. 2019/0343732, which is herein incorporated by reference in its entirety, leave-on oral care compositions, jammed emulsions, dentifrice compositions, mouth rinse compositions, mouthwash compositions, tooth gel, subgingival gel, mouth rinse, mousse, foam, mouth spray, lozenge, chewable tablet, chewing gum, tooth whitening strips, floss and floss coatings, breath freshening dissolvable strips, denture care products, denture adhesive products, or combinations thereof.

Oral Care Regimen

The dicarboxylic acid can be delivered in the same composition as the tin and/or fluoride or the dicarboxylic acid can be delivered in a separate composition. For example, a first composition can comprise tin and/or fluoride and a second composition can comprise dicarboxylic acid. The first and second composition can be delivered simultaneously, such as in a dual-phase composition or sequentially from discrete compositions.

An oral care kit can include the first composition comprising tin and/or fluoride and the second composition comprising dicarboxylic acid. The oral care kit can also include instructions directing a user to apply the first composition to an oral cavity of the user followed by applying the second composition to the oral cavity of the user. The first composition can be expectorated prior to the application of the second composition or the second composition can be applied prior to the expectoration of the first composition from the oral cavity.

The entire oral care regimen can have a duration of from one minute to about three minutes with each application step having a duration of from about 30 seconds to about 2 minutes or about 1 minute.

The components can be delivered to the oral cavity simultaneously or sequentially. The simplest case is simultaneous, continuous delivery of equal amounts of the two components or a constant ratio of the components during a single oral care session. The two components may be provided separately, such as in a dual-phase composition in two separate compositions, and then delivered simultaneously to the oral cavity. Brushing duration is sufficiently short so that the components will not be inactivated. Another use for simultaneous, continuous delivery is systems that include two components that react relatively slowly, and that will remain in the oral cavity after brushing to be absorbed by the teeth and or gums.

In the case of sequential delivery, both components may be delivered during a single oral care session, e.g., a single brushing session or other single treatment session (single use, start to finish, by a particular user, typically about 0.1 to 5 minutes), or alternatively the components may be delivered individually over multiple oral care sessions. Many combinations are possible, for example delivery of both components during a first oral care session and delivery of only one of the components during a second oral care session.

Sequential delivery during a single oral care session may take various forms. In one case, two components are delivered in alternation, as either a few relatively long duration cycles during brushing (A B A B), or many rapid-fire alternations (A B A B A B A B A B . . . A B).

In another case, two or more components are delivered one after the other during a single oral care session, with no subsequent alternating delivery in that oral care session (A followed by B). For example, a first composition comprising fluoride and/or tin can be delivered initially, to initiate brushing and provide cleansing, followed by a second composition comprising dicarboxylic acid.

### EXAMPLES

The invention is further illustrated by the following examples, which are not to be construed in any way as imposing limitations to the scope of this invention. Various other aspects, modifications, and equivalents thereof which, after reading the description herein, may suggest themselves to one of ordinary skill in the art without departing from the spirit of the present invention or the scope of the appended claims.

TABLE 1A
Oral Care Compositions

| Ingredient (wt %) | Ex. 1 | Ex. 2 | Ex. 3 | Ex. 4 |
|---|---|---|---|---|
| Sorbitol | 45.0000 | 47.0000 | 48.0000 | 48.0000 |
| Treated Water | 19.1091 | 20.2620 | 19.6550 | 21.1311 |
| SnF₂ | 0.4540 | — | — | 0.4540 |
| SnCl₂ (10% silica blend) | 0.5619 | — | — | 0.5619 |
| NaF | — | 0.2430 | — | — |
| Sodium Gluconate | 1.3000 | — | — | 1.3000 |
| NaOH 50% | 0.1500 | — | — | 0.8700 |
| Saccharin | 0.3500 | 0.3500 | 0.3500 | 0.4000 |
| Sucralose | 0.0800 | 0.0800 | 0.0800 | 0.2000 |
| Xanthan Gum | 0.8750 | 0.8750 | 0.8750 | 0.8750 |
| Carrageenan | 1.5000 | 1.5000 | 1.5000 | 1.5000 |
| Citric Acid | — | 0.2750 | 0.1250 | — |
| Zinc Citrate | — | — | — | 0.5330 |
| Na Citrate | 1.2050 | — | — | — |
| Potassium oxalate | 3.1400 | 3.1400 | 3.1400 | — |
| monohydrate | | | | |
| TiO₂ | 0.5000 | 0.5000 | 0.5000 | 0.5000 |
| Silica | 17.5000 | 17.5000 | 17.5000 | 17.5000 |
| Sodium Lauryl Sulfate | 7.0000 | 7.0000 | 7.0000 | 5.0000 |
| (28 wt % solution) | | | | |
| Flavor | 1.2750 | 1.2750 | 1.2750 | 1.1750 |

TABLE 1B
Oral Care Compositions

| Ingredient (wt % ) | Ex. 5 |
|---|---|
| Flavor | 1.20% |
| Sodium Monofluorophosphate | 1.15% |
| Sorbitol Solution (70%) | 49.90% |
| Mica-Titanium Dioxide coated | 0.50% |
| Cocamidopropyl Betaine Solution (30%) | 1.50% |
| Potassium Oxalate | 3.00% |
| Silica Thickening | 1.50% |
| Silica Abrasive | 12.00% |
| Sodium lauryl sulfate solution (28%) | 5.50% |
| Sodium saccharin | 0.40% |
| Sucralose powder | 0.08% |
| Phosphoric Acid | 0.55% |
| Xanthan Gum | 0.75% |
| Carrageenan Iota | 1.50% |
| Water | 20.50% |

TABLE 2
Summary of Tested Oral Care Compositions

| Examples | Summary of Ingredients |
|---|---|
| Ex. 1 | SnF₂, SnCl₂, Oxalate |
| Ex. 2 | NaF, Oxalate |
| Ex. 3 | Oxalate |
| Ex. 4 | SnF₂, SnCl₂ |
| Ex. 5 | MFP, Oxalate |
| Crest ® Cavity Protection (CCP) | NaF |
| Crest ® GumCare (CGC) | SnF₂ |

The treatment compositions included those from TABLE 1A and the summary TABLE 2. Ex. 1 included stannous fluoride, stannous chloride, and potassium oxalate (a dicarboxylic acid). Ex. 2 was similar to Ex. 1 except Ex. 2 replaced stannous fluoride/stannous chloride with sodium fluoride. Ex. 3 removes sodium fluoride from Ex. 2. Ex. 4 is the same as Ex. 1, but without potassium oxalate.

The enamel softening treatment compositions included those from TABLE 1B. Ex. 5 was compared to water (negative) and citric acid (positive) softening controls.

In Vitro Pellicle Tea Stain Model

The in vitro pellicle tea stain model (PTSM) is a technique in which plaque biomass is grown on glass rods from pooled human stimulated saliva over the course of three days. The plaque biomass is treated with agents, e.g. dentifrice supernatant, to determine the chemical staining potential. The purpose of this technique is to provide a simple, quick and reliable method for determining the effect of compounds on the amount of dental plaque stain.

Polished rods were treated with saliva supplemented with sucrose (0.1% w/v, 0/N), supplemented broth culture [trypticase soy broth (15 g), sucrose (50 g), and deionized water (467 ml) with freshly pooled saliva (33 g) (5 hr)] and freshly pooled saliva (0/N). The plaque growth was treated three times with 25% (w/v) dentifrice supernatant (5 min) and tea (10 min) [500 ml hot DI water with 5 Lipton tea bags steeped for 5 min, cooled to ˜22° C.] with at least one hour incubation in freshly pooled saliva between treatments. After the last treatment, rods were placed in freshly pooled saliva (O/N). Plaque was dried, weighed, and digested in KOH (0/N). Digested suspension was filtered (0.45 μm) and read for absorbance at 380 nm.

Glass rods were weighed to the nearest 0.0001 g and placed into specially designed holders. The position of each rod was then carefully adjusted to ensure that all rods would receive the same treatment exposure when dipping in solutions. Rod holders were then placed into a 37° C. incubator fitted with an automated dipping mechanism. The dipping mechanism was designed to dip rods 1.5 cm into 16×75 mm glass test tubes then completely remove the rods from the test tubes at a rate of one revolution per minute (one dip per minute). Four (4) rods were used for each treatment group.

In the afternoon of day 1, pooled saliva (400 ml) was supplemented with sucrose (0.40 g). 7 ml of this saliva was dispensed into dipping tubes. The dipping tubes were placed in 37° C. incubator and dipped to a depth of 15 mm at a rate of 1 rpm. The mixture was incubated overnight.

In the morning of day 2, broth was supplemented with freshly pooled saliva (33 g) and 7 ml supplemented broth mixture was dispensed into each dipping tube. The dipping tubes were placed in 37° C. incubator and dipped to a depth of 15 mm at a rate of 1 rpm. The dipping tubes were incubated for at least 5 hours. After 5 hours, broth culture tubes were replaced with tubes containing 7 mL of freshly pooled saliva. The tubes were placed in 37° C. incubator and dipped to a depth of 15 mm at a rate of 1 rpm. The tubes were incubated overnight.

In the morning of day 3, slurries were prepared (1:3 dentifrice:pooled saliva) using the compositions of TABLE 2. The slurries were centrifuged at 15,000 rpm for 15 minutes, decanted and the supernatant was isolated. Deionized water (500 ml) was heated in a microwave, high setting, for 10 minutes. A magnetic stir bar was added and 5 tea bags (Lipton) were allowed to steep for 5 minutes. 7 mL of freshly pooled saliva was dispensed into new tubes, 7 mL of supernatant into representative tubes, 7 ml of tea into new tubes, and 10 ml of deionized water were added into each of two rows of rinse tubes behind treatment and tea. Samples were treated for 5 minutes then rinsed by dipping up and down into deionized water 10 times for each row. After the final dip into DI water, the base of the rods was touched against a plastic cover to catch drips and the rods were inserted into tea for 10 minutes. This procedure was repeated for all rows of rods and incubated for 1 hour. These steps were repeated two additional times, followed by an overnight incubation in saliva

In the morning of day 4, saliva was removed and the rods were dried for 1 hour. The biomass on each rod was weighed. Each rod was individually placed into 3 mL 0.5M KOH in a test tube and incubated at 37° C. without dipping/shaking overnight.

In the morning of day 5, samples were removed from the incubator and allowed to cool to ˜22° C. The test tube was vortex mixed for 5 seconds, and each rod was removed from its respective test tube. 200 μL of dissolved plaque was transferred into wells of a 96 well plate and absorbance of the plaque digest was measured at 380 nm against 0.5M KOH as the blank in a plate reader (Spectra Max M2, Molecular Devices).

To analyze the pellicle stain potential of the tested formulations, absorbance values at A_{380 nm} were normalized for plaque growth and reported as [A_{380 nm}/mg Biomass]. Staining potential was also reported as [% Stain] compared to Crest® Gum Care (100%) and Crest® Cavity Protection (0%).

TABLE 3
Stain Accumulation of in Vitro Oral Biofilms determined by PTSM

| | | Stain | | |
|---|---|---|---|---|
| | Summary of | (A380/mg | | Staining |
| Examples | Ingredients | Biomass) | SD | Potential |
| Ex. 1 | SnF₂, SnCl₂, | 1.82 | 0.069 | 23.21 |
| | Oxalate | | | |
| Ex. 2 | NaF, Oxalate | 1.33 | 0.093 | −24.14 |
| Ex. 3 | Oxalate | 1.23 | 0.059 | −34.63 |
| Ex. 4 | SnF₂, SnCl₂ | 1.81 | 0.123 | 22.12 |
| CCP | NaF | 1.58 | 0.068 | 0.00 |
| CGC | SnF₂ | 2.61 | 0.047 | 100.00 |

TABLE 3 shows the staining potential, reported as the % stain compared to Crest® Gum Care (100%, can lead to cationic antimicrobial agents stains) and Crest® Cavity Protection (0%, did not include a cationic antimicrobial agent), of the compositions of TABLE 3. Ex. 4 (SnF₂/SnCl₂) showed a stain potential of 22.12%. Ex. 1 (SnF₂/SnCl₂+Oxalate) showed a stain potential of 23.31, which indicated that the stain left by Ex. 1/Ex. 4 was due to the use of a cationic antimicrobial agent, such as tin ions. According to TABLE 3, the stain from cationic antimicrobial agents were not impacted by the inclusion of oxalate. However, Ex. 2 and Ex. 3 showed unexpected improvements in stain removal caused by tea. For example, Ex. 2 (NaF+Oxalate) showed a 24% additional improvement in stain removal compared with CCP, which did not include a cationic antimicrobial agent. Additionally, Ex. 3 (Oxalate with no F) showed a 34% additional improvement in stain removal compared with CCP, which was unexpected.

Pellicle Cleaning Ratio (PCR)

The method of the pellicle cleaning ratio (PCR) is a well-accepted industry method to investigate the whitening properties of abrasive-containing compositions as a means to estimate their clinical stain removal potential. The method was originally published by Stookey et al. (1982) and was later refined by Schemehorn et al. (2011) to make a darker, more tenacious stain. The method of Schemehorn et al. was used here to evaluate the ability of the oxalate-containing formulations to remove a dental stain mimic. Their stain removal efficacy was determined as a PCR value which is the relative amount of cleaning that a test formulation produced relative to the control suspension of calcium pyrophosphate in a thickened slurry, again described in detail by Stookey et al. and Schemehorn et al. The PCR values obtained herein are reported in TABLE 4. The statistical grouping was determined using a student's t-test with α=0.05 using the JMP statistical software package. Treatments with different letter codes are statistically significantly different, p<0.05.

TABLE 4
PCR Values

| | Summary of | |
|---|---|---|
| Examples | Ingredients | PCR |
| Ex. 1 | SnF₂, SnCl₂, Oxalate | 141 ± 20 |
| Ex. 2 | NaF, Oxalate | 163 ± 23 |
| Ex. 3 | Oxalate | 146 ± 21 |
| Ex. 4 | SnF₂, SnCl₂ | 109 ± 16 |
| Ex. 5 | MPF, Oxalate | 139 ± 12 |
| CCP | NaF | 84 ± 10 |
| Crest ProHealth | SnF₂, Sodium | 140 ± 22 |
| Advanced Whitening | Hexametaphosphate | |

TABLE 4 shows the cleaning efficacy of a variety of oral care compositions as illustrated by the PCR value. The PCR is calculated by comparing the before and after images of a brushed stained-bovine chip. A higher PCR value indicates that more stain was removed.

Crest ProHealth Advanced Whitening (CPHAW) is a whitening toothpaste including stannous fluoride and sodium hexametaphosphate (whitening/anticalculus agent) and was used as the positive control in this experiment. CCP is a toothpaste included sodium fluoride, but no whitening/Anticalculus agent and was used as the negative control in this experiment. CPHAW had a PCR value of about 140 while CCP had a PCR value of about 84. Ex. 4 (SnF₂/SnCl₂) had a PCR value of 109. However, unexpectedly, the addition of oxalate to Ex. 1 resulted in a PCR value of 141. Importantly, Ex. 2 (NaF+Oxalate) and Ex. 3 (Oxalate with no F) also displayed far higher PCR values than the negative control with 163 and 146, respectively. The results indicate a significant stain removal potential directly attributed to the oxalate.

Desirable compositions include those with a PCR value of at least about 120, 130, 140, 150, and/or 160, as determined by the PCR method described herein. Desirable compositions also include those where the PCR value of the oxalate-containing composition is about 1.2, about 1.3, or about 1.4× greater than the pH-matched, oxalate-placebo of the same composition.

Powder Stain Removal Model (PSRM)

The PSRM is a screening technique where hydroxyapatite powder (HAP) is used as a substrate for stain accumulation (see Baig A A; Kozak K M; Cox E R et al; J Clin Dent 2002, 13:19 24). The purpose of this technique was to illustrate and quantify the stain removal properties of chemical agents used in the oral care compositions disclosed herein with respect to pH. Hydroxyapatite powder provides a large surface area to which tea chromogens adsorbed. In addition to the published PSRM a modification of the model was used where tea stain was used in conjunction with Iron Trichloride (FeCl3) to simulate stains that are more resistant to removal. Stains found in the oral cavity commonly contain Fe(III) (Tantbirojn D, Douglas W H et al Eur J Oral Sci 1998; 106,971-976).

Treatment of stained HAP with oral care compositions resulted in different levels of stain removal depending upon the ability of the actives to disrupt the binding of these chromogens to the HAP surface. The magnitude of stain removal was quantified by image analysis.

A tea solution was prepared using 8 regular teabags (Lipton Black Tea) in 400 mL boiling water and steeping for 5 min. After letting the tea cool to 50° C. it was filtered through a Nalgene sterile filter (Thermo Scientific Nalgene Rapid flow 75 mm Filter Unit, 0.2SCFA). 10 g Hydroxyapatite Powder (HAP, Bio-Gel HTP-Gel Catalog #130-0421, Bio-Rad Laboratories Hercules, Calif.) was added and the mixture was stirred for 5 min, distributed into 12 50-mL centrifuge tubes and centrifuged for 15 min at 14,000 rpm (Lynx 6000, Thermo Fischer with Lynx F14-14X50CY rotor). Supernatant was decanted, the remaining stained HAP-powder was washed twice with water by adding 25 ml of water, vortexing, centrifuging at 14,000 rpm for 15 mins, and decanting liquid. Centrifuge tubes were placed in a convection oven overnight at 60° C. to dry stained HAP. Once dried, stained HAP was pooled and ground to a fine powder with pestle and mortar and stored in a dry dark place at room temperature.

For tea solutions that were modified to include iron, a tea solution was prepared as described above and after filtering, 2.0 g Iron (III) Chloride (Ferric Chloride Hexahydrate; VWR BDH9234) was added under stirring. Then HAP-powder was added, powder was centrifuged, washed and dried to yield a deeply colored powder.

Treatment solutions were prepared for oxalate and pyrophosphate anions. Oxalate solutions (0.34M, 3%) of different pH (4.5, 5.0, 5.5, 7) were obtained by mixing appropriate ratios of potassium oxalate (62.79 g/L Potassium Oxalate Monohydrate in water, pH-7.0) and oxalic acid solution (30.69 g Oxalic Acid/L in water, pH-1.3). Lower concentrations were obtained by further dilution with water. A pyrophosphate solution, equimolar to a 3% Oxalate solution (75.65 g/L Sodium acid pyrophosphate, pH-adjusted with IN NaOH to pH7) was used as comparison.

HAP powders were treated with the treatment solutions to investigate their stain removal potential. Treatment (20 mL of a 1:4 dilution of treatment solutions above) was added to stained HAP powder (200 mg) in a 50 mL centrifuge, vortexed for 1 min, then centrifuged at 14,000 rpm for 15 min. Supernatant was decanted and the pellet was washed twice by adding 25 mL of water, vortexing, centrifuging at 14,000 rpm for 15 mins, and decanting. The twice washed pellet was resuspended in 30 mL water by vortexing for 1 min, then vacuum-filtered (Millipore MF Membrane Filters 8.0 um, 47 mm) to collect the powder. The filter cake was dried overnight, then laminated between approximately 3″ squares of self-adhesive laminating sheets (Avery 9″×12″ #73601 Dennison Brea, Ca 92821). Treatments were done in triplicate. A control disk using tea-stained HAP from the same batch treated with water was prepared as well.

The color of the laminated samples was measured using a color-calibrated, white-balanced digital camera and RGB values were acquired. The camera had a lens equipped with a polarizer filter (Camera model no. CANON EOS 70D from Canon Inc., Melville, N.Y. with NIKON 55 mm micro-NIKKOR lens with adapter). The light system was equipped with Dedo lights (model number DLH2) equipped with 150 watt, 24V bulbs model number (Xenophot model number HL X64640), positioned about 30 cm apart (measured from the center of the external circular surface of one of the glass lens through which the light exits to the other) and aimed at a 45 degree angle such that the light paths. A sample holder was used to fix the laminated sheets perpedicular to the camera and acquire reproducible images. The obtained RGB-values were converted into CIE-LAB values using imaging software (Optimas, Mediacybernetics).

Stain removal was determined as change in L (brightness) vs. untreated stained HAP, calculated as ΔL=(L for treated HAP-L for the untreated stained HAP). The PSRM stain removal data are given below in TABLE 5.

TABLE 5
PSRM Stain Removal Data.

| | | % | Tea Stain | Tea Stain + |
|---|---|---|---|---|
| pH | Active | Active | (ΔL) | FeCl₃ (ΔL) |
| 4.5 | Oxalate | 3.00 | 10.79 | 5.66 |
| | Solution | 1.00 | 5.67 | 3.73 |
| | | 0.50 | 3.41 | 0.20 |
| | | 0.10 | 3.36 | −0.55 |
| 5 | Oxalate | 3.00 | 5.76 | 3.68 |
| | Solution | 1.00 | 4.42 | 0.93 |
| | | 0.50 | 2.51 | −0.24 |
| | | 0.10 | 0.70 | −1.50 |
| 5.5 | Oxalate | 3.00 | 2.92 | 0.15 |
| | Solution | 1.00 | 2.47 | 0.10 |
| | | 0.50 | 1.83 | −1.11 |
| | | 0.10 | 0.13 | −2.03 |
| 7 | Oxalate | 3.00 | 2.78 | −0.81 |
| | Solution | 1.00 | 2.11 | −0.97 |
| | | 0.50 | 1.28 | −0.91 |
| | | 0.10 | 0.38 | −2.08 |
| 7 | Pyrophosphate | 7.56.00 | 9.85 | 2.88 |
| | Solution* | 2.5200 | 7.88 | 0.77 |
| | | 1.250 | 5.97 | 0.02 |
| | | 0.250 | 5.16 | −0.97 |

*Equimolar to Oxalate Solution

The data in Table 5 show that Oxalate in concentrations above 0.5% for tea-stain and 1% for Fe(III)-containing stain has stain removal efficacy which increases with lower pH. Desirable compositions as described herein include compositions with a ΔL value of at least about 2, at least about 3, at least about 5, or at least about 10.

Enamel Softening

The enamel softening method is used to determine the potential of oral care compositions to damage (or not to damage) dental enamel with repeated exposure. A microhardness tester was used to determine the change in hardness of dental enamel following cyclic exposure to the oral care compositions in TABLE 1A and TABLE 1B, in comparison to the control compositions: 1) deionized water; and 2) 1% citric acid solution.

A core of sound human enamel with a diameter of 3-4 mm was extracted from whole human teeth. The cores were mounted in dental acrylic and the surfaces were ground using 600 grit paper. Increasingly fine lapping papers were then used to polish the surface to a 1μ polish. Samples were sonicated in deionized water for 30 min. Enamel specimens were then rinsed with deionized water and wiped to remove any residual polish. Each enamel specimen was inspected and samples with large cracks or uneven calcification were discarded. Enough specimens were prepared to provide 8 specimens for each treatment group. Enamel specimens were stored in an airtight container above a small amount of deionized water (˜1-5 mL) in a standard laboratory refrigerator (˜2-4° C.).

The artificial saliva solution of TABLE 6 was prepared on the day before the experiment. Also on the day before the experiment, the Vickers hardness of each enamel specimen was measured using a hardness indenter at three separate locations spread across the enamel surface. A 50 g load was applied for 10 seconds, and the diagonal lengths of the resulting indents were measured using a 20× magnification objective. The average Vickers hardness of the three indents was used to determine the average pre-cycling enamel hardness. Enamel specimens were then assigned to treatment groups such that the average hardness of each treatment group and the standard deviation of the average hardness were similar.

TABLE 6
Artificial Saliva Solution

| | | Molecular | Target | Target | |
|---|---|---|---|---|---|
| Materials | Formula | Weight | Molarity | (1 Liter) | |
| Calcium Nitrate, Tetrahydrate | Ca(NO₃)₂ * 4H₂O | 236.15 | Ca 0.8 | 0.3540 | g |
| Potassium Phosphate | KH₂PO₄ | 136.09 | 2.4 | 0.1230 | g |
| Potassium Chloride | KCl | 74.55 | 130 | 11.18 | g |
| BisTris (CAS 6976-37-0) | C₈H₁₉NO₅ | 209.24 | 20 | 4.185 | g |

| | | | | |
|---|---|---|---|---|
| Hydrochloric Acid, Concentrated | HCl | 36.46 | — | Adjust to pH 7 |
| Deionized Water | H₂O | — | — | 1000 ml |

On the day of the cycling treatments, each treatment group was removed from the storage container and rinsed. The samples were cycled for a total of six rounds through the following procedure:
1) Specimens were treated by group in a 1:3 well-mixed slurry of toothpaste to water under quiescent conditions. The control group specimens were treated with deionized water or 1% citric acid solution.
2) The specimens were rinsed with copious amounts of water until residual toothpaste was removed.
3) The specimens were treated in quiescent saliva for 55 minutes.
4) The specimens were rinsed with copious amounts of water until residual saliva was removed.

Following the sixth round of this exposure protocol, the specimens were stored in an airtight container over, but not touching, a small amount of deionized water.

On the day following the cycling experiment, the post-cycling hardness was obtained for each specimen using a similar procedure to that described for the pre-cycling hardness measurements. The change in hardness was calculated for each specimen by subtracting the pre-cycling hardness from the post-cycling hardness measurement. The average change in specimen hardness with respect to treatment and its standard deviation were then determined.

The statistical grouping was then determined using JMP with an α=0.05 in a student's t-test. The cycling was repeated if the average change in specimen hardness for the 1% citric acid positive control was not significantly different from the deionized water negative control. Statistical significance was checked for the difference between the dentifrice-slurry-treated specimens and those in the negative control, deionized water, treatment group. Those treatments that were significantly different than the negative control were determined to detrimentally soften the enamel surface.

The results of the enamel softening experiment are given in TABLE 7. At pH ca. 4.5 an oxalate version of a low pH toothpaste was found to damage enamel relative to the water negative control. Because of these data, the pH range of oxalate-containing toothpastes to prevent softening of the enamel surface can be at least about 4.5.

TABLE 7
Enamel Softening Results Showing
Change in Surface Microhardness (ΔSMH).

| | Slurry | | Statistical | |
|---|---|---|---|---|
| Treatments | pH | Δ SMH | Grouping | Study |
| Water | 5.3 | 15.02 | C | 1 |
| 1% Citric Acid | 2.19 | −175.68 | A | 1 |
| Ex. 5 | 4.56 | −42.42 | B | 1 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A whitening dentifrice composition comprising:
(a) dicarboxylic acid; and
(b) a pH of from about 4 to about 6,
wherein the whitening dentifrice composition has a pellicle cleaning ratio of at least about 120.

2. The composition of claim 1, wherein the dicarboxylic acid comprises a compound with the formula HO₂C—R—CO₂H.

3. The composition of claim 2, wherein R is aliphatic, aromatic, or combinations thereof.

4. The composition of claim 1, wherein the dicarboxylic acid comprises oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, maleic acid, malic acid, tartaric acid, phthalic acid, methylmalonic acid, dimethylmalonic acid, tartronic acid, mesoxalic acid, dihydroxymalonic acid, fumaric acid, terephthalic acid, salts thereof, or combinations thereof.

5. The composition of claim 1, wherein the dicarboxylic acid comprises oxalic acid, malonic acid, malic acid, maleic acid, phthalic acid, or combinations thereof.

6. The composition of claim 1, wherein the pH is from about 4.5 to about 6.

7. The composition of claim 1, wherein the composition comprises cationic antimicrobial agent.

8. The composition of claim 7, wherein the cationic antimicrobial agent comprises quaternary ammonium salt, metal ion, or combinations thereof.

9. The composition of claim 8, wherein the metal ion comprises tin.

10. The oral care composition of claim 9, wherein the tin comprises stannous fluoride, stannous chloride, or combinations thereof.

11. The oral care composition of claim 1, wherein the oral care composition comprises fluoride.

12. The oral care composition of claim 11, wherein the fluoride comprises stannous fluoride, sodium fluoride, sodium monofluorophosphate, amine fluoride, or combinations thereof.

13. The oral care composition of claim 1, wherein the oral care composition comprises polyphosphate.

14. The oral care composition of claim 13, wherein the polyphosphate comprises pyrophosphate, tripolyphosphate, tetrapolyphosphate, hexametaphosphate, or combinations thereof.

15. The oral care composition of claim 1, wherein the oral care composition is free of, essentially free of, or substantially free of polyphosphate.

16. The oral care composition of claim 1, wherein the oral care composition comprises zinc.

17. The oral care composition of claim 16, wherein the zinc comprises zinc citrate, zinc lactate, zinc oxide, zinc phosphate, or combinations thereof.

18. The oral care composition of claim 1, wherein the oral care composition is free of, essentially free of, or substantially free of zinc.

19. The oral care composition of claim 1, wherein the oral care composition comprises monodentate ligand, polydentate ligand, or combinations thereof.

20. The oral care composition of claim 19, wherein the oral care composition has a tin to monodentate ligand to polydentate molar ratio of from about 1 to 0.5 to 0.5 to about 1 to 5 to 5.

21. The oral care composition of claim 1, wherein the oral care composition comprises thickening agent.

22. The oral care composition of claim 21, wherein the thickening agent comprises polysaccharide, polymer, silica thickener, or combinations thereof.

23. The oral care composition of claim 1, wherein the oral care composition comprises abrasive.

24. The oral care composition of claim 23, wherein the abrasive comprises silica abrasive, calcium abrasive, or combinations thereof.

25. The oral care composition of claim 24, wherein the silica abrasive comprises precipitated silica.

26. The oral care composition of claim 24, wherein the calcium abrasive comprises calcium carbonate, calcium pyrophosphate, calcium phosphate, hydroxyapatite, or combinations thereof.

27. The oral care composition of claim 1, wherein the oral care composition comprises amino acid.

28. The oral care composition of claim 27, wherein the amino acid comprises basic amino acid, acidic amino acid, neutral amino acid, or combinations thereof.

29. The oral care compositions of claim 28, wherein the amino acid comprises glycine, alanine, valine, isoleucine, tryptophan, phenylalanine, proline, methionine, leucine, serine, threonine, tyrosine, asparagine, glutamine, cysteine, citrulline, aspartic acid, glutamic acid, lysine, arginine, histidine, or combinations thereof.

30. The oral care composition of claim 1, wherein the oral care composition comprises whitening agent.

31. The oral care composition of claim 30, wherein the whitening agent comprises peroxide, polyphosphate, or combinations thereof.

32. The oral care composition of claim 1, wherein the oral care composition comprises humectant.

33. The oral care composition of claim 32, wherein the humectant comprises glycerin, sorbitol, erythritol, xylitol, butylene glycol, propylene glycol, polyethylene glycol, or combinations thereof.

34. The oral care composition of claim 1, wherein the oral care composition comprises no added water.

35. The oral care composition of claim 1, wherein the oral care composition comprises water.

36. The oral care composition of claim 35, wherein the oral care composition comprises up to 45%, by weight of the composition, of water.

37. The composition of claim 1, wherein the composition has a PCR value of at least about 140.
